SEQUENCE LISTING (12 sequences)

SEQ ID NO: 1 - protein, 331 aa, Streptomyces mobaraensis
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 2 - protein, 382 aa, Artificial sequence
  Recombinant transglutaminase proenzyme
DNGAGEETKSYAETYRLTADDVANINALNESAPAASSAGPSFRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWPHHHHHH

SEQ ID NO: 3 - protein, 341 aa, Artificial sequence
  Activated recombinant transglutaminase
  MISC_FEATURE (1)..(4): N-Terminal tetrapeptide
  MISC_FEATURE (336)..(341): C-Terminal polyhistidine
FRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWPHHHHHH

SEQ ID NO: 4 - protein, 331 aa, Artificial sequence
  Variant M8
  MISC_FEATURE (300)..(300): E300A Substitution
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSAGYSDFDRGAYVITFIPKSWNTAPDKVKQGWP

SEQ ID NO: 5 - protein, 331 aa, Artificial sequence
  Variant M10
  MISC_FEATURE (240)..(240): I240A Substitution
  MISC_FEATURE (241)..(241): P241A Substitution
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNAARSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 6 - protein, 331 aa, Artificial sequence
  Variant M12
  MISC_FEATURE (249)..(249): E249Q Substitution
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGQGFVNFDYGWFGAQTEADADKTVWTHGNHYHAPNGSLGAMHVYESKFRNWSEGYSDFDRGAYVITFIPKSWNTAPDKVKQGWP

SEQ ID NO: 7 - protein, 331 aa, Artificial sequence
  Variant M14
  MISC_FEATURE (300)..(300): E300A Substitution
  MISC_FEATURE (302)..(302): Y302A Substitution
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSAGASDFDRGAYVITFIPKSWNTAPDKVKQGWP

SEQ ID NO: 8 - protein, 4 aa, Artificial sequence
  N-terminal tetrapeptide
FRAP

SEQ ID NO: 9 - protein, 9 aa, Artificial sequence
  Trypsin digest fragment
  MISC_FEATURE (3)..(3): Antibody Q295 residue
  MISC_FEATURE (5)..(5): Antibody N297 residue
EEQYNSTYR

SEQ ID NO: 10 - DNA, 66 bp, Artificial sequence
  Primer zg67,901
tagaaataattttgtttaactttaagaaggagatatatatatggataacggcgcgggcgaagaaac

SEQ ID NO: 11 - DNA, 91 bp, Artificial sequence
  Primer zg67,900
tctgtatcaggctgaaaatcttatctcatccgccaaaacattagtgatggtgatggtgatgtgaacccggccagccctgtttcactttatc

SEQ ID NO: 12 - DNA, 1238 bp, Artificial Sequence
  Variant M8 amplicon
tagaaataattttgtttaactttaagaaggagatatatatatggataacggcgcgggcgaagaaaccaaaagctatgcggaaacctatcgcctgaccgcggatgatgtggcgaacattaacgcgctgaacgaaagcgcgccggcggcgagcagcgcgggcccgagctttcgcgcgccggatagcgatgatcgcgtgaccccgccggcggaaccgctggatcgcatgccggatccgtatcgcccgagctatggccgcgcggaaaccgtggtgaacaactatattcgcaaatggcagcaggtgtatagccatcgcgatggccgcaaacagcagatgaccgaagaacagcgcgaatggctgagctatggctgcgtgggcgtgacctgggtgaacagcggccagtatccgaccaaccgcctggcgtttgcgagctttgatgaagatcgctttaaaaacgaactgaaaaacggccgcccgcgcagcggcgaaacccgcgcggaatttgaaggccgcgtggcgaaagaaagctttgatgaagaaaaaggctttcagcgcgcgcgcgaagtggcgagcgtgatgaaccgcgcgctggaaaacgcgcatgatgaaagcgcgtatctggataacctgaaaaaagaactggcgaacggcaacgatgcgctgcgcaacgaagatgcgcgcagcccgttttatagcgcgctgcgcaacaccccgagctttaaagaacgcaacggcggcaaccatgatccgagccgcatgaaagcggtgatttatagcaaacatttttggagcggccaggatcgcagcagcagcgcggataaacgcaaatatggcgatccggatgcgtttcgcccggcgccgggcaccggcctggtggatatgagccgcgatcgcaacattccgcgcagcccgaccagcccgggcgaaggctttgtgaactttgattatggctggtttggcgcgcagaccgaagcggatgcggataaaaccgtgtggacccatggcaaccattatcatgcgccgaacggcagcctgggcgcgatgcatgtgtatgaaagcaaatttcgcaactggagcgcgggctatagcgattttgatcgcggcgcgtatgtgattacctttattccgaaaagctggaacaccgcgccggataaagtgaaacagggctggccgggttcacatcaccatcaccatcactaatgttttggcggatgagataagattttcagcctgatacaga

(19) United States
(12) Patent Application Publication
(10) Pub. No.: US 2018/0265851 A1
(43) Pub. Date: Sep. 20, 2018

(54) TRANSGLUTAMINASE VARIANTS FOR CONJUGATING ANTIBODIES

(71) Applicant: BRISTOL-MYERS SQUIBB COMPANY, Princeton, NJ (US)

(72) Inventors: Chetana RAO-NAIK, Walnut Creek, CA (US); Ganapathy N. SARMA, San Ramon, CA (US); Akbar NAYEEM, Newtown, PA (US)

(21) Appl. No.: 15/763,677

(22) PCT Filed: Sep. 30, 2016
(86) PCT No.: PCT/US2016/054585

§ 371 Date: Mar. 27, 2018

Related U.S. Application Data
(60) Provisional application No. 62/236,274, filed on Oct. 2, 2015.

(51) Int. Cl. C12N 9/10; C12P 21/00; A61K 47/68

(52) U.S. Cl. CPC C12N 9/1044; A61K 47/68; C12P 21/00

(57) ABSTRACT

Transglutaminase variants capable of conjugating an antibody that is not conjugated by wild-type transglutaminase from Streptomyces mobaraensis.

### CROSS-REFERENCE TO RELATED APPLICATIONS

This application claims the benefit under 35 U.S.C. § 119(e) of U.S. Provisional Application No. 62/236,274, filed Oct. 2, 2015; the disclosure of which is incorporated herein by reference.

### SEQUENCE LISTING

Incorporated herein by reference in its entirety is a Sequence Listing named “12610WOPCT_ST25,” comprising SEQ ID NO:1 through SEQ ID NO:12, which include nucleic acid and/or amino acid sequences disclosed herein. The Sequence Listing has been submitted herewith in ASCII text format via EFS-Web, and thus constitutes both the paper and computer readable form thereof. The Sequence Listing was first created using PatentIn 3.5 on Sep. 24, 2015, and is approximately 25 KB in size.

### BACKGROUND OF THE INVENTION

Antibodies have many applications in medicine and biotechnology. For some applications, it is desirable to conjugate the antibody with another chemical moiety, that is, covalently attaching the antibody to such moiety. The moiety can be, for instance, another protein, a radioisotope, an assay agent (e.g., biotin or a fluorescent label), or a drug.

Many methods having been disclosed for effecting conjugation. The enzyme transglutaminase, in particular bacterial transglutaminase from Streptomyces mobaraensis, having an amino acid sequence according to SEQ ID NO:1 and referred to hereinafter as BTG, has been used to conjugate antibodies and other proteins

BTG can form an amide bond between the carboxamide side chain of a glutamine (the amine acceptor) in a first protein and the ε-amino group of a lysine (the amine donor) in a second protein, in a transamidation reaction. Specificity-wise, it is selective regarding the glutamine residue, requiring that it be located in a flexible part of a protein loop and flanked by particular amino acids. Conversely, BTG is permissive regarding the lysine residue: it even accepts an amino group from a non-protein source, such an alkyleneamino compound, as a lysine ε-amino surrogate. See Fontana et al. 2008.

Antibodies of the IgG isotype have many glutamines—nine or more in the heavy chain constant region alone, the exact number depending on isotype. However, none of them are BTG-reactive in a native antibody—that is, they are not transamidated by transglutaminase—and some modification of the antibody is necessary to induce reactivity. Normally, an antibody is glycosylated at asparagine 297 (N297) of the heavy chain (N-linked glycosylation). Jeger 2009 and Jeger et al. 2010 disclosed that deglycosylation of the antibody, either by eliminating the glycosylation site through an N297A substitution or post-translation enzymatic deglycosylation with an enzyme such as s PNGase F (peptide-N-glycosidase F), renders nearby glutamine 295 (Q295) BTG-reactive. (References to amino acid positions in an antibody constant region employ numbering per the EU index as set forth in Kabat et al., “Sequences of proteins of immunological interest,” 5th ed., Pub. No. 91-3242, U.S. Dept. Health & Human Services, NIH, Bethesda, Md., 1991; hereinafter “Kabat.”) They further showed that an N297Q substitution not only eliminates glycosylation, but also introduces a second glutamine residue, at position 297, that is an amine acceptor. Thus, simple deglycosylation generates two BTG-reactive glutamine residues per antibody (one per heavy chain, at Q295), while an N297Q substitution generates four BTG-reactive glutamine residues (two per heavy chain, at Q295 and Q297).

The glutamine selectivity of BTG can be modulated by altering its amino acid sequence. Working with human growth hormone (hGH), Norskov-Lauritsen et al. 2009 found that the selectivity of BTG for Gln-40 compared to Gln141 in hGH can be improved by replacing up to three basic or acidic amino acid residues with other basic or acidic amino acids. Working with a different organism, Streptoverticillium ladakanum, Hu et al. 2009, 2010a, and 2010b reported that the selectivity of its transglutaminase for Gln-141 could be increased by modifying its amino acid sequence at certain positions or by adding residues to its N-terminus.

Tagami et al. 2009 and Yokoyama et al. 2010 have studied the effect of mutations on the specific activity of BTG against the dipeptide N-carbobenzoxy-L-glutaminylglycine (and also ovalbumin in the case of Tagami et al. 2009) as an amine acceptor. The substitutions they made and their effects on specific activity are summarized in Table 1 and Tables 2-4 thereof, respectively. See also Rao-Naik, U.S. Provisional Application Ser. No. 62/236,282, filed Oct. 2, 2015, for another disclosure on glutamine-containing tags.

In an approach complementary to modifying the amino acid sequence of BTG to alter its substrate specificity or activity, the structure of an antibody can be modified to make it BTG-reactive. In addition to the modifications disclosed by Jeger 2009 and Jeger et al. 2010, discussed above, a glutamine-containing peptide, or “tag,” can be added to an antibody to introduce an exogenous glutamine that is BTG-reactive. See Dorywalska et al. 2015; Pons et al. 2013 and Rao-Naik 2015. The tag can be a glutamine inserted or substituted into the antibody—that is, a single amino acid insertion or substitution—or the tag can be a glutamine-containing polypeptide inserted at the N-terminus, middle, or C-terminus of an antibody chain, commonly but not necessarily the heavy chain.

Among the antibody conjugates, one type that is generating strong interest in the medical field is an antibody-drug conjugate (ADC, also referred to as an immunoconjugate). In an ADC, a therapeutic agent (also referred to as the drug, payload, or warhead) is covalently linked to an antibody whose antigen is expressed by a cancer cell (tumor associated antigen). The antibody, by binding to the antigen, delivers the ADC to the cancer site. There, cleavage of the covalent link or degradation of the antibody leads to the release of the therapeutic agent. Conversely, while the ADC is circulating in the blood system, the therapeutic agent is held inactive because of its covalent linkage to the antibody. Due to its localized release, the therapeutic agent in an ADC can be much more potent (cytotoxic) than ordinary chemotherapy agents. In summary, an ADC comprises three components: (1) an antibody, (2) a drug, and (3) a linker covalently joining the antibody and the drug. For a review on ADCs generally, see Schrama et al. 2006. Disclosures relating to the BTG-mediated preparation of ADCs include: Dennler et al. 2014, Hu et al. 2015, Innate Pharma 2013, Jeger 2009, Jeger et al. 2010, Lhospice et al. 2015, Pons et al. 2013, and Strop et al., 2013.

Other transglutaminase disclosures, generally relating to the labeling or modification of proteins (including antibodies), include: Bregeon 2014, Bregeon et al. 2013 and 2014, Chen et al. 2005, Fischer et al. 2014, Kamiya et al. 2011, Lin et al. 2006, Mero et al. 2009, Mindt et al. 2008, Sato 2002, Sato et al. 2001, Schlibi et al. 2007, and Sugimura et al. 2007.

Full citations for the documents cited herein by first author or inventor and year are listed at the end of this specification.

### BRIEF SUMMARY OF THE INVENTION

While in principle BTG is an attractive agent for making an antibody conjugate, a practical limitation is the need to modify the antibody in some manner—deglycosylation or adding a tag containing a BTG receptive glutamine—so that a glutamine is available as an amine acceptor.

The present invention provides variant transglutaminases and methods for using them to make an antibody conjugate. The methods of this invention are not limited to any particular antibody, but they are especially advantageously used to conjugate an antibody in its native state, i.e., one that has not been modified to introduce an exogenous BTG-reactive glutamine or to render an endogenous glutamine BTG-reactive and is not conjugatable with BTG.

In a first aspect, the present invention provides a method of making an antibody conjugate, comprising:
(a) mixing an antibody with an amine donor compound comprising a primary amine and a moiety selected from the group consisting of a protein, a radioisotope, an assay agent, and a drug, in the presence of a variant transglutaminase comprising an amino acid sequence that is at least 90% identical (preferably at least 95% identical and more preferably 100% identical) to SEQ ID NO:1, with the proviso that the variant transglutaminase has an amino acid substitution feature selected from the group consisting of (A) E300A, (B) I240A and P241A, (C) E249Q, and (D) E300A and Y302A; and
(b) allowing the variant transglutaminase to catalyze the formation of an amide bond between the side chain carboxamide of a glutamine of the antibody and the primary amine of the amine donor compound, thereby making the antibody conjugate.

In a second aspect, the present invention provides another method of making an antibody conjugate, comprising:
(a) mixing an antibody with a first compound, which first compound is an amine donor compound having a primary amine and a first reactive functional group, in the presence of a variant transglutaminase comprising an amino acid sequence that is at least 90% identical (preferably at least 95% identical and more preferably 100% identical) to SEQ ID NO:1, with the proviso that the variant transglutaminase has an amino acid substitution feature selected from the group consisting of (A) E300A, (B) I240A and P241A, (C) E249Q, and (D) E300A and Y302A;
(b) allowing the variant transglutaminase to catalyze the formation of an amide bond between the side chain carboxamide of a glutamine of the antibody and the primary amine of the first compound, to make an adduct of the antibody and the first compound;
(c) contacting the adduct with a second compound having a second reactive functional group and a moiety selected from the group consisting of a protein, a radioisotope, an assay agent, and a drug; the second reactive functional group being capable of reacting with the first reactive functional group to form a covalent bond therebetween; and
(d) allowing the first and second reactive functional groups to react and form a covalent bond therebetween, thereby making the antibody conjugate.

In either of the two preceding methods, the antibody preferably is an IgG antibody having a glutamine at position 295 (Q295) and a glycosylated asparagine at position 297 (N297), numbering per the EU index as in Kabat. Such an antibody is not transamidated by BTG, but is transamidated at Q295 by the variant transglutaminases of this invention.

In either of the preceding two methods, the variant transglutaminase preferably has an amino acid substitution feature from the group consisting of (B) I240A and P241A, (C) E249Q, and (D) E300A and Y302A.

Where moiety (in the first compound or second compound, as the case may be) is a protein, the resultant conjugate is a fusion protein. Where the moiety is a radioisotope, the resultant conjugate can be used for radiation therapy. The moiety can be an assay agent such as a fluorescent label or a ligand like biotin, in which case the conjugate can be used for diagnostic or analytical applications. Preferably, the moiety is a drug, in which case the product is an antibody-drug conjugate, which can be used in medical treatments, especially the treatment of cancer.

In yet another aspect, this invention provides a variant transglutaminase comprising an amino acid sequence that is at least 90% identical (preferably at least 95% identical and more preferably 100% identical) to SEQ ID NO:1, with the proviso that said variant transglutaminase has an amino acid substitution feature selected from the group consisting of (a) I240A and P241A, (b) E249Q, and (c) E300A and Y302A. In one preferred embodiment, the substitution feature is I240A and P241A. In another preferred embodiment, the substitution feature is E249Q. In yet another preferred embodiment, the substitution feature is E300A and Y302A.

### BRIEF DESCRIPTION OF THE DRAWING(S)

FIG. 1 shows schematically the BTG mediated preparation of a conjugate, via the two processes respectively referred to as the one-step and the two-step process.

FIG. 2 is a western blot showing the results of conjugation of various antibodies with the transglutaminase variant designated as M8.

FIGS. 3A and 3B compare the trypsin digest fragments of an anti-glypican 3 antibody alone and conjugated using variant M8.

FIG. 4 is a western blot of antibodies conjugated with transglutaminase variants designated as M10, M12, and M14, along with results for two comparative/control antibodies.

### DETAILED DESCRIPTION OF THE INVENTION

The variant transglutaminases of this invention are capable of conjugating an antibody that is not reactive towards S. mobaraensis transglutaminase. This is a significant advantage, as the need to engineer or modify the antibody in some manner is avoided.

One transglutaminase variant of this invention, designated M8, has a single mutation (E300A), relative to the sequence of the wild-type S. mobaraensis transglutaminase (SEQ ID NO:1). The amino acid sequence of variant M8 is shown in SEQ ID NO: 4. Tagami et al. 2009 disclosed, among over 30 microbial transglutaminase variants, an E300A variant, but only evaluated it for specific activity against CBZ-Gln-Gly or ovalbumin.

Another transglutaminase variant of this invention, designated M10, has a double mutation (I240A and P241A), relative to the sequence of the wild-type S. mobaraensis transglutaminase (SEQ ID NO:1). The amino acid sequence of variant M10 is shown in SEQ ID NO: 5.

Yet another transglutaminase variant of this invention, designated M12, has a single mutation (E249Q), relative to the sequence of the wild-type S. mobaraensis transglutaminase (SEQ ID NO:1). The amino acid sequence of variant M12 is shown in SEQ ID NO: 6.

Yet another transglutaminase variant of this invention, designated M12, has a double mutation (E300A and Y302A), relative to the sequence of the wild-type S. mobaraensis transglutaminase (SEQ ID NO:1). The amino acid sequence of variant M10 is shown in SEQ ID NO: 7.

Variants M8, M10, M12, and M14 can have conservative substitutions thereto, provided their respective distinctive substitutions (a) E300A, (b) I240A/P241A, (c) E249Q, or (d) E300A/Y302A are preserved. Such conservatively modified versions of variants M8, M10, M12, and M14 are included in the scope of this invention. A “conservative modification” or “conservative substitution” means, in respect of a polypeptide, the replacement of an amino acid therein with another amino acid having a similar side chain. Families of amino acids having similar side chains are known in the art. Such families include amino acids with basic side chains (lysine, arginine, histidine), acidic side chains (aspartic acid, glutamic acid), uncharged polar side chains (asparagine, glutamine, serine, threonine, tyrosine, cysteine, tryptophan), nonpolar side chains (glycine, alanine, valine, leucine, isoleucine, proline, phenylalanine, methionine), beta-branched side chains (threonine, valine, isoleucine), small side chains (glycine, alanine, serine), chain orientation changing side chains (glycine, proline) and aromatic side chains (tyrosine, phenylalanine, tryptophan). Plural conservative substitutions/modifications may be present. Preferably, conservatively modified versions of variants M8, M10, M12, and M14 are at least 90% identical, more preferably at least 95% identical to their respective unmodified sequences, or, alternatively, have between 1 and 3 conservative amino acid substitutions.

BTG variants M8, M10, M12, and M14 may further comprise an N-terminal extension of a tetrapeptide according to SEQ ID NO:8 (FRAP). Yokoyama et al. 2010 disclose, in the context of an S199A substitution, that an FRAP extension may positively affect specific activity.

BTG variants M8, M10, M12, and M14 may further comprise a polyhistidine peptide extension at their C-terminus, as exemplified with amino acid residues 336-441 of SEQ ID NO:3. The polyhistidine peptide is a useful tag for purification purposes and does not affect enzymatic activity. Typically, the polyhistidine peptide is 6-8 residues long, preferably six residues long.

Antibodies that can be conjugated by the methods of this invention include those recognizing the following antigens: mesothelin, prostate specific membrane antigen (PSMA), CD19, CD22, CD30, CD70, B7H3, B7H4 (also known as O8E), protein tyrosine kinase 7 (PTK7), glypican-3, RG1, fucosyl-GM1, CTLA-4, and CD44. The antibody can be animal (e.g., murine), chimeric, humanized, or, preferably, human. The antibody preferably is monoclonal, especially a monoclonal human antibody. The preparation of human monoclonal antibodies against some of the aforementioned antigens is disclosed in Korman et al., U.S. Pat. No. 8,609,816 B2 (2013; B7H4, also known as 08E; in particular antibodies 2A7, 1G11, and 2F9); Rao-Naik et al., U.S. Pat. No. 8,097,703 B2 (2012; CD19; in particular antibodies 5G7, 13F1, 46E8, 21D4, 21D4a, 47G4, 27F3, and 3C10); King et al., U.S. Pat. No. 8,481,683 B2 (2013; CD22; in particular antibodies 12C5, 19A3, 16F7, and 23C6); Keler et al., U.S. Pat. No. 7,387,776 B2 (2008; CD30; in particular antibodies 5F11, 2H9, and 17G1); Terrett et al., U.S. Pat. No. 8,124,738 B2 (2012; CD70; in particular antibodies 2H5, 10B4, 8B5, 18E7, and 69A7); Korman et al., U.S. Pat. No. 6,984,720 B1 (2006; CTLA-4; in particular antibodies 10D1, 4B6, and 1E2); Vistica et al., U.S. Pat. No. 8,383,118 B2 (2013, fucosyl-GM1, in particular antibodies 5B1, 5B1a, 7D4, 7E4, 13B8, and 18D5) Korman et al., U.S. Pat. No. 8,008,449 B2 (2011; PD-1; in particular antibodies 17D8, 2D3, 4H1, 5C4, 4A11, 7D3, and 5F4); Huang et al., US 2009/0297438 A1 (2009; PSMA. in particular antibodies 1C3, 2A10, 2F5, 2C6); Cardarelli et al., U.S. Pat. No. 7,875,278 B2 (2011; PSMA; in particular antibodies 4A3, 7F12, 8C12, 8A11, 16F9, 2A10, 2C6, 2F5, and 1C3); Terrett et al., U.S. Pat. No. 8,222,375 B2 (2012; PTK7; in particular antibodies 3G8, 4D5, 12C6, 12C6a, and 7C8); Terrett et al., U.S. Pat. No. 8,680,247 B2 (2014; glypican-3; in particular antibodies 4A6, 11E7, and 16D10); Harkins et al., U.S. Pat. No. 7,335,748 B2 (2008; RG1; in particular antibodies A, B, C, and D); Terrett et al., U.S. Pat. No. 8,268,970 B2 (2012; mesothelin; in particular antibodies 3C10, 6A4, and 7B1); Xu et al., US 2010/0092484 A1 (2010; CD44; in particular antibodies 14G9.B8.B4, 2D1.A3.D12, and 1A9.A6.B9); Deshpande et al., U.S. Pat. No. 8,258,266 B2 (2012; IP10; in particular antibodies 1D4, 1E1, 2G1, 3C4, 6A5, 6A8, 7C10, 8F6, 10A12, 10A12S, and 13C4); Kuhne et al., U.S. Pat. No. 8,450,464 B2 (2013; CXCR4; in particular antibodies F7, F9, D1, and E2); and Korman et al., U.S. Pat. No. 7,943,743 B2 (2011; PD-L1; in particular antibodies 3G10, 12A4, 10A5, 5F8, 10H10, 1B12, 7H1, 11E6, 12B7, and 13G4); the disclosures of which are incorporated herein by reference.

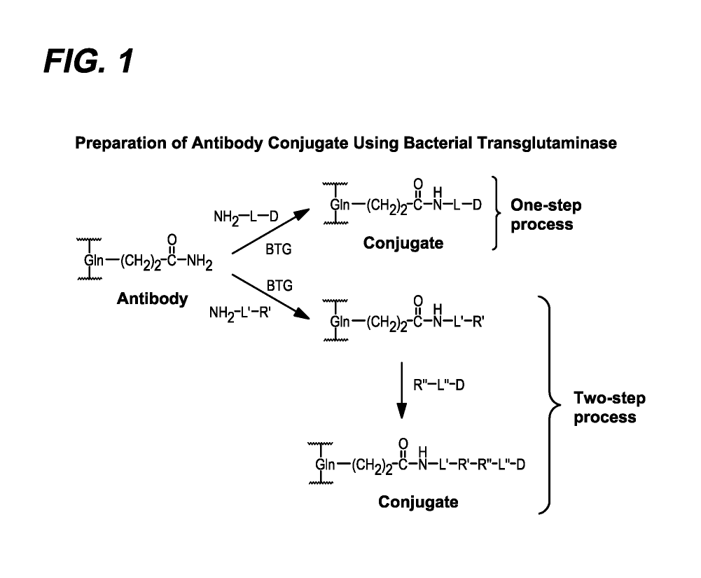

BTG-mediated preparation of an antibody conjugate can be by a one-step process or a two-step process, as illustrated schematically in FIG. 1. In the one-step process, BTG couples a glutamine carboxamide on the antibody acting as the amine acceptor and an amine donor compound H2N-L-D, where L is a linker moiety and D is a protein, a radioisotope, an assay agent, or a drug, to form the conjugate directly. In the two-step process, BTG catalyzes the formation of an initial transamidation adduct between an antibody glutamine carboxamide acting as the amine receptor and first compound H2N-L′-R′, which is an amine donor compound, where L′ is a linker moiety and R′ is a first reactive functional group. Subsequently, the adduct is reacted with a second compound R″-L″-D, where R″ is a second reactive functional group capable of reacting with R′, L″ is a linker moiety, and D is as defined above. Sometimes, the one-step process is referred to as the enzymatic process, and the two-step process as the chemo-enzymatic process.

The amine donor, whether H2N-L-D or H2N-L′-R′, is often used in large excess to suppress undesired transamidation between the glutamine carboxamide and an ε-amino group of an antibody lysine. If the moiety D is expensive or difficult to obtain, the use of a large excess may be impractical. In such instances, the two-step process may be preferable.

In a preferred embodiment, amine donor compound in a one-step process is represented by formula (I):

H₂N⁻(CH₂)₂₋₆D (I)

where D is a protein, a radioisotope, an assay agent, or a drug.

More preferably, the one-step method is used to make an ADC, so that the amine donor compound can have a structure represented by formula (Ia):

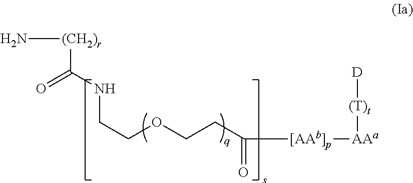

wherein
D is a drug;
T is a self-immolating group;
t is 0 or 1;
AA^{a} and each AA^{b} are independently selected from the group consisting of alanine, β-alanine, γ-aminobutyric acid, arginine, asparagine, aspartic acid, γ-carboxyglutamic acid, citrulline, cysteine, glutamic acid, glutamine, glycine, histidine, isoleucine, leucine, lysine, methionine, norleucine, norvaline, ornithine, phenylalanine, proline, serine, threonine, tryptophan, tyrosine, and valine;
p is 1, 2, 3, or 4;
q is 1, 2, 3, 4, 5, 6, 7, 8, 9, or 10;
r is 1, 2, 3, 4, or 5; and
s is 0 or 1.

In formula (Ia), -AA^{a}-[AA^{b}]ₚ- represents a polypeptide whose length is determined by the value of p (dipeptide if p is 1, tetrapeptide if p is 3, etc.). AA^{a} is at the carboxy terminus of the polypeptide and its carboxyl group forms a peptide (amide) bond with an amine nitrogen of drug D (or self-immolating group T, if present). Conversely, the last AA^{b} is at the amino terminus of the polypeptide and its α-amino group forms a peptide bond with

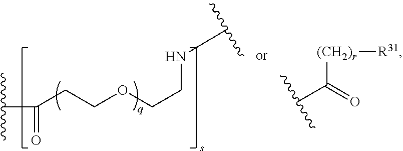

depending on whether s is 1 or 0, respectively. Preferred polypeptides -AA^{a}-[AA^{b}]ₚ- are Val-Cit, Val-Lys, Lys-Val-Ala, Asp-Val-Ala, Val-Ala, Lys-Val-Cit, Ala-Val-Cit, Val-Gly, Val-Gln, and Asp-Val-Cit, written in the conventional N-to-C direction, as in H₂N-Val-Cit-CO₂H). More preferably, the polypeptide is Val-Cit, Val-Lys, or Val-Ala. Preferably, a polypeptide -AA^{a}-[AA^{b}]ₚ- is cleavable by an enzyme found inside the target (cancer) cell, for example a cathepsin and especially cathepsin B.

If the subscript s is 1, drug-linker (Ia) contains a poly(ethylene glycol) (PEG) group, which can advantageously improve the solubility of drug-linker (Ia), facilitating conjugation to the antibody—a step that is performed in aqueous media. Also, a PEG group can serve as a spacer between the antibody and the peptide -AA^{a}-[AA^{b}]ₚ-, so that the bulk of the antibody does not sterically interfere with action of a peptide-cleaving enzyme.

As indicated by the subscript t equals 0 or 1, a self-immolating group T is optionally present. A self-immolating group is one such that cleavage from AA^{a} or AA^{b}, as the case may be, initiates a reaction sequence resulting in the self-immolating group disbonding itself from drug D and freeing the latter to exert its therapeutic function. When present, the self-immolating group T preferably is a p-aminobenzyl oxycarbonyl (PABC) group, whose structure is shown below, with an asterisk (*) denoting the end of the PABC bonded to an amine nitrogen of drug D and a wavy line ( [symbol] ) denoting the end bonded to the polypeptide -AA^{a}-[AA^{b}]ₚ-.

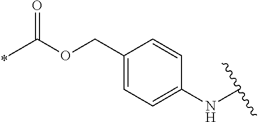 [SMILES: *Nc1ccc(COC(C)=O)cc1]

Another self-immolating group that can be used is a substituted thiazole, as disclosed in Feng, U.S. Pat. No. 7,375,078 B2 (2008).

In a two-step conjugation, many combinations of groups R′ and R″ can be used. Suitable combinations of R′ and R″ (or, vice-versa, R″ and R′) include:
(a) a maleimide group and a sulfhydryl group, to form a Michael addition adduct, as in

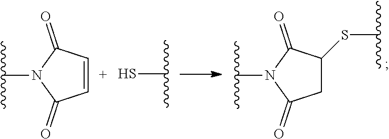

(b) a dibenzocyclooctyne group and an azide group, to form a cycloaddition product via “click” chemistry, as in

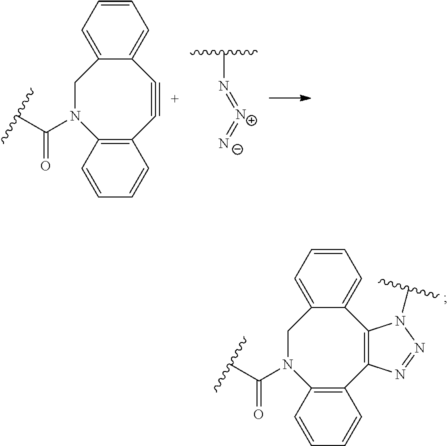

(c) an N-hydroxysuccinimide ester and an amine, to form an amide, as in

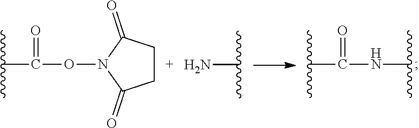

and
(d) an aldehyde or ketone (where “alkyl” preferably is C₁₋₃ alkyl) and a hydroxylamine, to form an oxime, as in

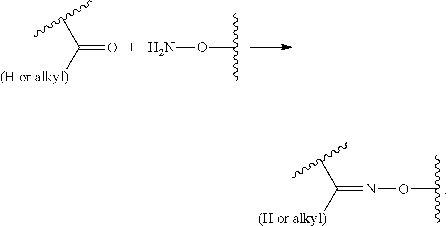

Thus, R′ can be selected from

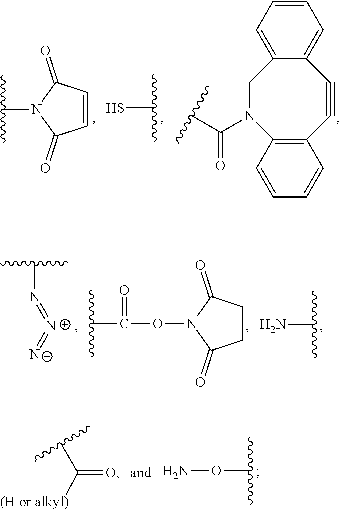

while, reciprocally, R″ can be selected from

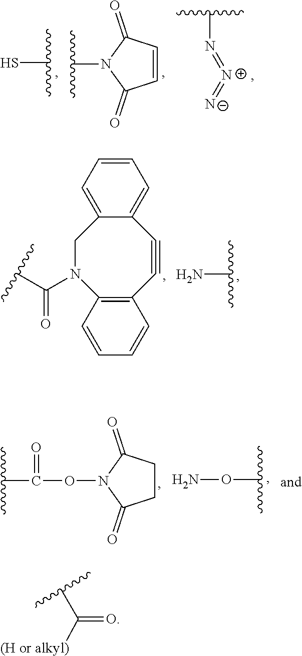

A suitable amine donor compound H₂N-L′-R′ for the two-step process is depicted in formula (II)

H₂N—(CH₂)₂₋₈—R′ (II)

where R′ is as defined above and preferably is

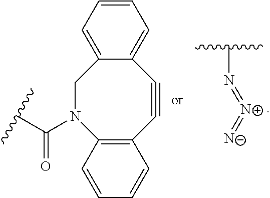

A corresponding suitable compound R″-L″-D is shown in formula (III)

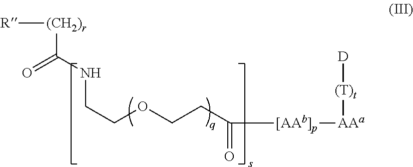

where R″ is as defined above and preferably is

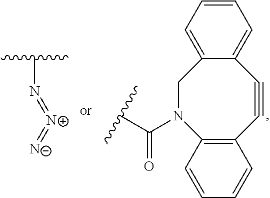

and r, q, s, AA^{b}, p, AA^{a}, T, t, and D are as defined above in respect of formula (Ia).

In the instance where the conjugate is an ADC intended for use in cancer treatment, the drug moiety preferably is a cytotoxic drug that causes death of the targeted cancer cell. Cytotoxic drugs that can be used in ADCs include the following types of compounds and their analogs and derivatives:
(a) enediynes such as calicheamicin (see, e.g., Lee et al., J. Am. Chem. Soc. 1987, 109, 3464 and 3466) and uncialamycin (see, e.g., Davies et al., WO 2007/038868 A2 (2007); Chowdari et al., U.S. Pat. No. 8,709,431 B2 (2012); and Nicolaou et al., WO 2015/023879 A1 (2015));
(b) tubulysins (see, e.g., Domling et al., U.S. Pat. No. 7,778,814 B2 (2010); Cheng et al., U.S. Pat. No. 8,394,922 B2 (2013); and Cong et al., U.S. Pat. No. 8,980,824 B2 (2015));
(c) DNA alkylators such as analogs of CC-1065 and duocarmycin (see, e.g., Boger, U.S. Pat. No. 6,5458,530 B1 (2003); Sufi et al., U.S. Pat. No. 8,461,117 B2 (2013); and Zhang et al., U.S. Pat. No. 8,852,599 B2 (2014));
(d) epothilones (see, e.g., Vite et al., US 2007/0275904 A1 (2007) and U.S. RE42930 E (2011));
(e) auristatins (see, e.g., Senter et al., U.S. Pat. No. 6,844,869 B2 (2005) and Doronina et al., U.S. Pat. No. 7,498,298 B2 (2009));
(f) pyrrolobezodiazepine (PBD) dimers (see, e.g., Howard et al., US 2013/0059800 A1 (2013); US 2013/0028919 A1 (2013); and WO 2013/041606 A1 (2013)); and
(g) maytansinoids such as DM1 and DM4 (see, e.g., Chari et al., U.S. Pat. No. 5,208,020 (1993) and Amphlett et al., U.S. Pat. No. 7,374,762 B2 (2008)).

Preferably, the drug is a DNA alkylator, tubulysin, auristatin, pyrrolobenzodiazepine, enediyne, or maytansinoid compound. Specific examples are:

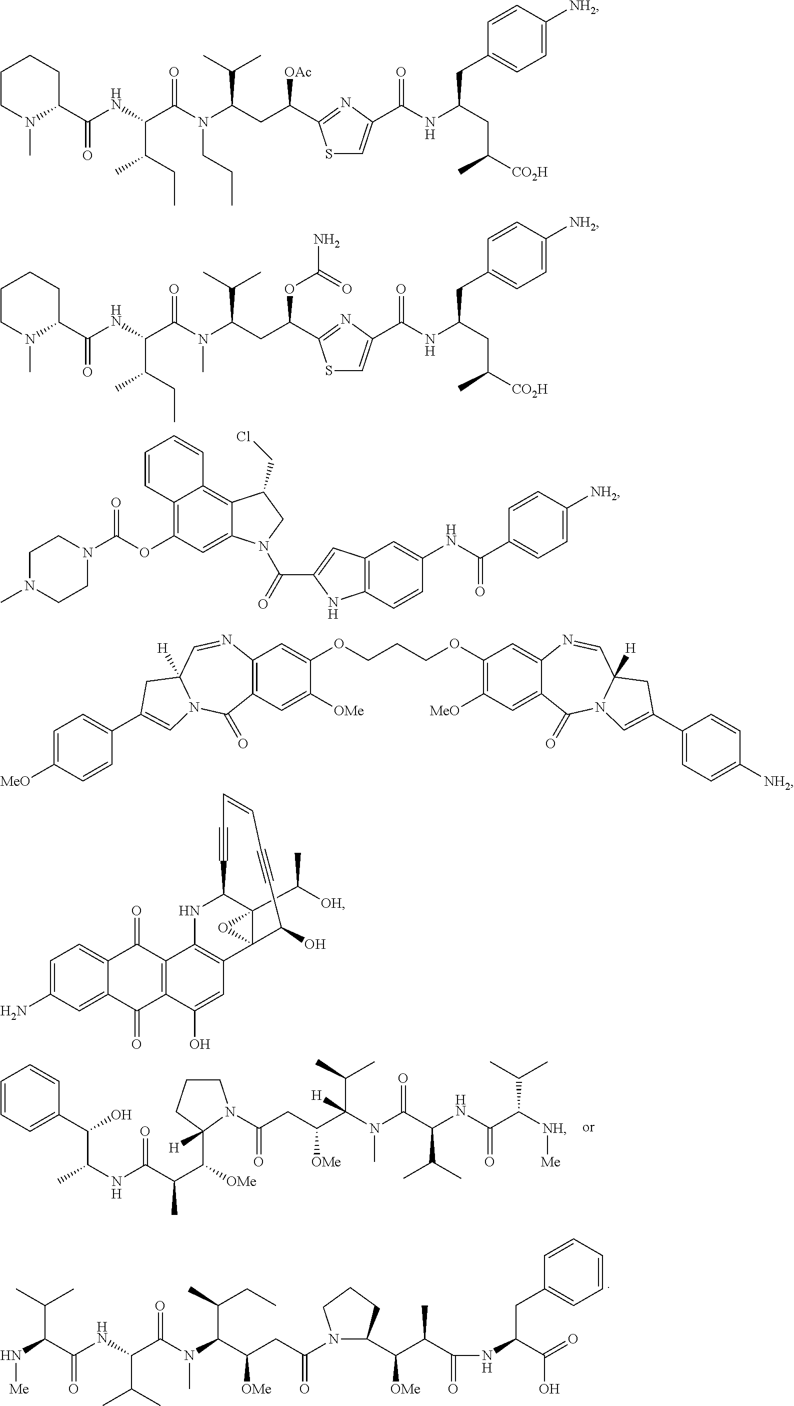

The functional group at which conjugation to the linker L or L″, as the case may be, is effected is the amine (—NH₂) group in the case of the first five drugs above and the methyl amine (—NHMe) group in the case of the last two drugs.

The aforementioned drug moieties can be used in ADCs made by either the one-step or two-step process.

The foregoing references, in addition to disclosing the drug moieties proper, also disclose drug-linker constructs according to formulae (Ia) or (III), or which can be readily adapted to make such drug-linker compounds, mutatis mutandis. Particularly pertinent disclosures relating to the preparation of drug-linker compounds are found in Chowdari et al., U.S. Pat. No. 8,709,431 B2 (2012); Cheng et al., U.S. Pat. No. 8,394,922 B2 (2013); Cong et al., U.S. Pat. No. 8,980,824 B2 (2015); Sufi et al., U.S. Pat. No. 8,461,117 B2 (2013); and Zhang et al., U.S. Pat. No. 8,852,599 B2 (2014). While these references may relate to specific drug moieties, those skilled in the art will appreciate that the principles of making drug-linker compounds there are applicable to other types of drugs, mutatis mutandis.

Those skilled in the art will appreciate that a special advantage in making antibody conjugates with the variant transglutaminases of this invention is the elimination of the need to modify the antibody to introduce an exogenous BTG-reactive glutamine or to render an endogenous glutamine BTG-reactive. However, the use of the variant transglutaminases to make conjugates of an antibody so modified is not precluded. An antibody can be modified to introduce a BTG-reactive exogenous antibody by substituting an endogenous amino acid with a glutamine. An N297Q substitution, as disclosed by Jeger 2009 and Jeget et al. 2010 is an example. Or, an exogenous glutamine can be introduced by inserting a glutamine containing peptide, or “tag,” at the N-terminus, the interior, or the C-terminus of the antibody (especially the heavy chain), as disclosed by Dorywaslka et al. 2015, Pons et al. 2013, and Rao-Naik 2015. An example of antibody modification to render an endogenous glutamine BTG-reactive is the activation of Q295 by eliminating glycosylation at position 297, by enzymatic deglycosylation, by an N297A substitution, or by an N297Q substitution, as disclosed by Jeger 2009 and Jeger et al. 2010.

A glutamine in an antibody is a BTG-reactive (synonymously, transglutaminase-reactive) glutamine if its carboxamide side chain acts as an amine acceptor for S. mobaraensis transglutaminase (SEQ ID NO:1), using hydroxylamine as the amine donor.

The practice of this invention can be further understood by reference to the following examples, which are provided by way of illustration and not of limitation.

### Example 1—Transglutaminase

The amino acid sequence of S. mobaraensis transglutaminase (BTG) is provided in SEQ ID NO:1. For generating the mutants of this invention, BTG was produced recombinantly by expression in E. coli, initially producing a proenzyme according SEQ ID NO:2. Activation by cleavage of an N-terminal peptide by dispase yielded recombinant BTG according to SEQ ID NO:3, which contained an FRAP tetrapeptide at the N-terminus and a polyhistidine tail at the C-terminus (amino acids 1-4 and 336-441 of SEQ ID NO:3, respectively). The core part of SEQ ID NO:3 (amino acids 5-335) was identical to SEQ ID NO:1. This recombinant BTG had the same activity as wild-type BTG. The preparation of recombinant BTG used herein is described in detail below.

Bacterial transglutaminase from S. mobaraensis was expressed in E. coli as a proenzyme with a C-terminal His-tag. Bacterial cell pellets expressing the proenzyme were collected and treated as follows: The pellet was weighed while frozen. For each 1 g of pellet, 2 mL of BPER II reagent, 0.5 mg/mL lysozyme, 0.5 U/mL BENZONASE® endonuclease (EMD Millipore), and one protease inhibitor tablet were added to re-suspend the pellet. After the re-suspension was homogenous, it was transferred to centrifuge tubes and centrifuged at 27000×g for 15 min. The supernatant was decanted into a separate container and extra re-suspension buffer was added to the pellet for further re-suspension and centrifuged at 27000×g for 15 minutes. This process was repeated twice and the collected supernatant fractions were pooled. The pooled supernatant fractions were filtered through a 0.2 μm filter before loading onto a column for purification.

A 5 mL HisTrap® Excel column was equilibrated with 50 mM tris-HCl, 300 mM NaCl, 2 mM CaCl₂, 1 mM glutathione, pH 8.0 for 10 CV. The extracted protein (˜40 mL) was loaded onto the column. The column was then washed with equilibration buffer (˜20 column volumes). The equilibration buffer with 1.3 mg/mL of dispase enzyme was then used to wash the column until baseline increased as an indication that dispase has been equilibrated within the column. The column was removed from the instrument and incubated at 37° C. for 1 h. Post incubation, the column was washed with equilibration buffer (without dispase) until baseline was reached. The activated protein was eluted with 35% Buffer B (50 mM Tris-HCl, 300 mM NaCl, 500 mM Imidazole pH 8.0).

The collected peak fractions from the elution were pooled and dialyzed overnight with 50 mM Na acetate, 500 mM NaCl pH 5.5. After dialysis, the final material was filtered through a 0.2 μm filter, aliquoted and stored at −80° C.

The Microbial Transglutaminase kit from Zedira was used to measure the specific activity of BTG and the variants of this invention. The kit uses N-carbobenzoxy-L-glutaminylglycine (Z-Gln-Gly or CBZ-Gln-Gly) as the amine acceptor substrate and hydroxylamine as amine donor. In the presence of transglutaminae, the hydoxylamine is incorporated to form Z-glutamylhydroxamate-glycine, which develops a colored complex with iron (III) detectable at 525 nm.

### Example 2—Preparation of Variant Transglutaminases

Transglutaminase inserts were amplified by PCR using recombination-specific primers zg67,901 (SEQ ID NO:10) and zg67,900 (SEQ ID NO:11). The primers were used to amplify a 1238 base pair transglutaminase fragment for each variant. Illustratively, the nucleotide sequence of the amplicon for variant M8 is provided in SEQ ID NO:12. Those skilled in the art will be able to derive the corresponding nucleotide sequences for variants M10, M12, and M14. The inserts were codon optimized in-house, include a C-terminal (His)6 tag and were used for subcloning into an inclusion body expression vector (pTAP238 acceptor vector, derived in-house). The resulting plasmids were designated pSDH839 (M8, E300A), pSDH835 (M10, I240A/P241A), pSDH836 (M12, E249Q) and pSDH840 (M14, E300A/Y302A). The plasmids were subsequently transformed into the E. coli host ZGOLDS for expression analysis and scale-up protein production.

### Example 3—Conjugations of Antibodies with Variant M8

N-(Biotinyl)cadaverine (NBC, obtained as its hydrochloride salt from Zedira GmbH, Germany, catalog #B002) was used as an amine donor compound to demonstrate the ability of BTG variants of this invention to conjugate antibodies at Q295, notwithstanding the presence of glycosylation at N297.

Transglutaminase variant M8 was used to conjugate NBC with four different antibodies (anti-mesothelin, anti-glypican 3, anti-fucosyl GM1, and anti-CD70, each of which was glycosylated at N297).

The anti-mesothelin and anti-glypican 3 runs were performed on a larger scale, as follows: Antibody was pre-diluted to 1.14 mg/mL for the conjugation reaction. For an 11.4 mg of antibody reaction (10 mL at 1.14 mg/mL), 0.255 mL of variant M8 (neat) and 1.126 mL of NBC (80-fold molar excess, 20-fold molar excess assuming four reactive glutamines per antibody) were added to the reaction mixture, resulting in a final antibody concentration of 1.0 mg/mL and final variant M8 concentration of 0.1 mg/mL. The reaction mixture was incubated for 24 hours at 37° C.

Conjugation reactions of the anti-fucosyl GM1 antibody were conducted on a smaller scale. Antibody was pre-diluted to 1.14 mg/mL. For a 0.57 mg antibody reaction (0.5 mL of 1.14 mg/mL), 0.0127 mL of variant M8 (neat) and 0.056 mL of NBC (80-fold molar excess payload, 20-fold molar excess per site) were added to the reaction mixture, resulting in a final antibody concentration of 1.0 mg/mL and final M8 concentration of 0.1 mg/mL. The reaction mixtures were incubated for 24 hours at 37° C.

After 24 hours incubation, the unconjugated biotin-cadaverine from the reaction mixture was cleaned up using MabSelect SuRe column. The column was first equilibrated with 1×PBS, pH 7.4 prior to loading. After loading the reaction mixture to the column, it was washed with equilibration buffer before eluting with 20 mM Glycine, 10 mM Succinate, pH 3.2. The elution pool was dialyzed overnight in formulation buffer (20 mg/mL Sorbitol, 10 mg/mL Glycine, pH 5.0).

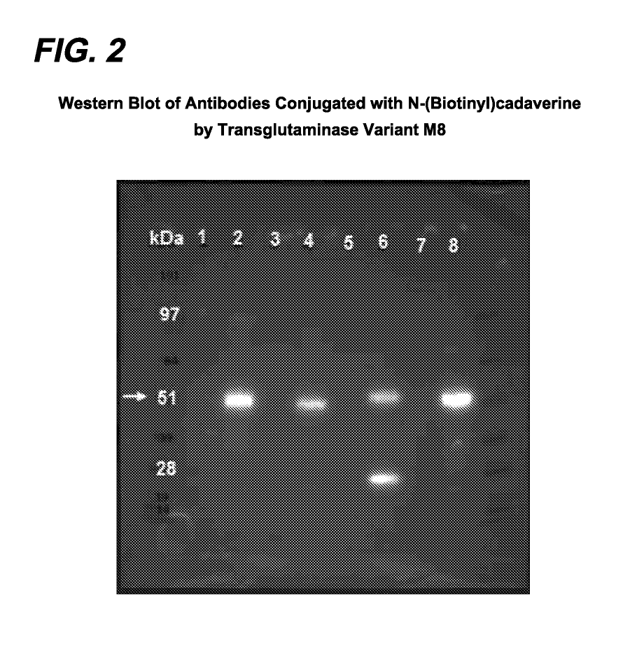

FIG. 2 is a western blot showing the conjugation results. NeutrAvidin Horseradish Peroxidase Conjugate (Thermo Scientific, Catalog #31001) was used to detect and visualize protein bound biotin by Neutravidin HRP. Table 1 shows the lane assignments.

TABLE 1
Conjugation of Antibodies to N-(Biotinyl)cadaverine
by Transglutaminase Variant M8

| | | N-(Biotinyl)- | Transglutaminase |
|---|---|---|---|
| Lane | Antibody | cadaverine | Variant M8 |
| 1 | Anti-mesothelin (control) | None | None |
| 2 | Anti-mesothelin | Yes | Yes |
| 3 | Anti-glypican 3 (control) | None | None |
| 4 | Anti-glypican 3 | Yes | Yes |
| 5 | Anti-fucosyl GM1 (control) | None | None |
| 6 | Anti-fucosyl GM1 | Yes | Yes |
| 7 | Anti-CD70 (control) | None | None |
| 8 | Anti-CD70 | Yes | Yes |

The 51 kDa band (arrow) corresponds to the heavy chain of the antibodies. Lanes 1, 3, 5, and 7, for the unconjugated antibodies, are dark. Conversely, the 51 kDa band is luminescent at lanes 2, 4, 6, and 8, evidencing that NBC was successfully conjugated to the antibody heavy chains. The anti-fucosyl GM1 antibody had a glutamine in CDR2 of its light chain, which apparently also was transamidated by BTG, accounting for the luminescent spot at 28 kDa in lane 6.

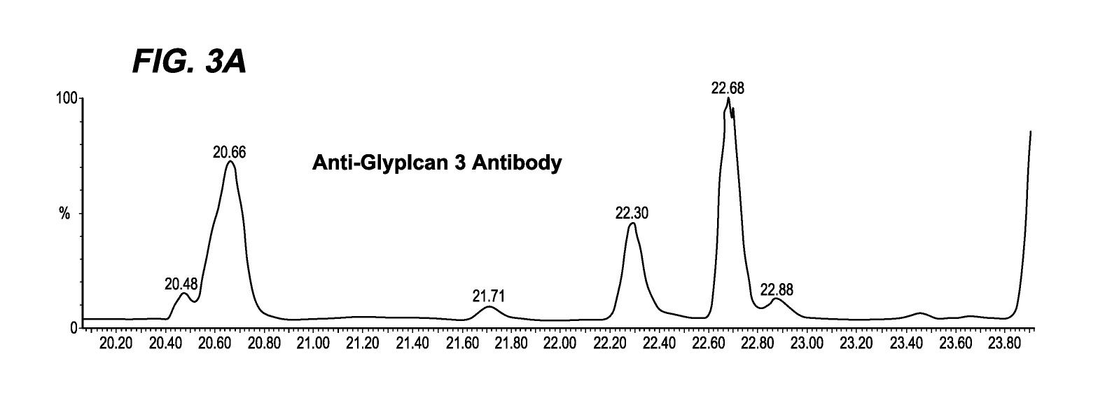

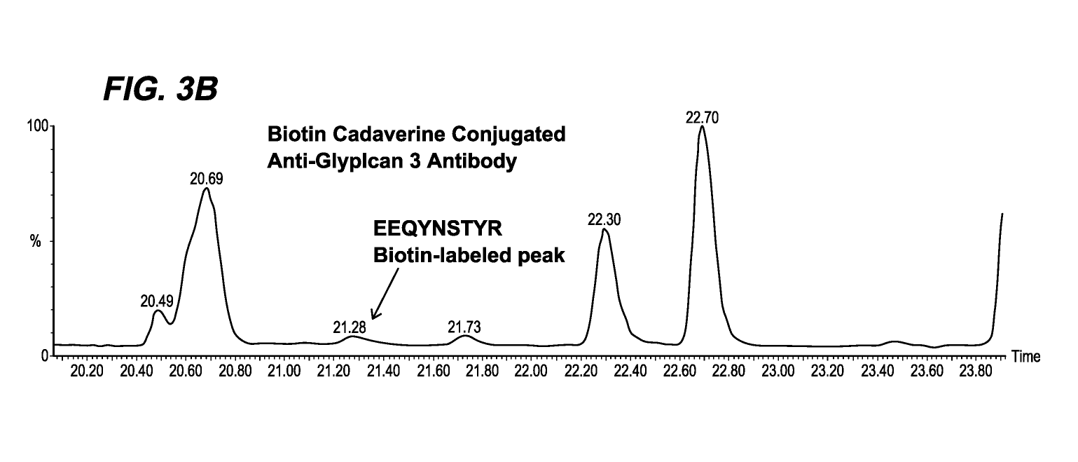

The anti-glypican 3 antibody used in the above experiments, both unconjugated and conjugated, was subjected to trypsin digestion. FIGS. 3A and 3B are chromatographic traces of the resulting fragments for the unconjugated and conjugated antibody, respectively. In FIG. 3B there is an additional peak corresponding to biotinylated peptide EEQYNSTYR (SEQ ID NO:9), pin-pointing Q295 as the glutamine transamidated by variant M8.

The number of biotin groups attached per antibody is shown in Table 2. Biotin content was measured using a Pierce Biotin Quantitation Kit from Thermo Scientific, which uses HABA (4′-hydroxyazobenzene-2-carboxylic acid) as the visualization reagent.

TABLE 2
Biotin to Antibody Ratio

| Conjugate | Biotin/Antibody Ratio |
|---|---|
| Anti-mesothelin antibody/biotin | 0.6 |
| Anti-glypican 3 antibody/biotin | 0.4 |
| Anti-fucosyl GM1 antibody/biotin | 0.9 |
| Anti-CD70 antibody/biotin | 0.6 |

### Example 4—Conjugation of Antibodies with Variants M10, M12, and M14

Using the same techniques as in the preceding example, transglutaminase variants M10, M12, and M14 were used to conjugate antibodies. Additionally, two other variants, designated M9 (Q74A/Y75F/P76G) and M11 (Y75F/N239A) were used as comparative examples.

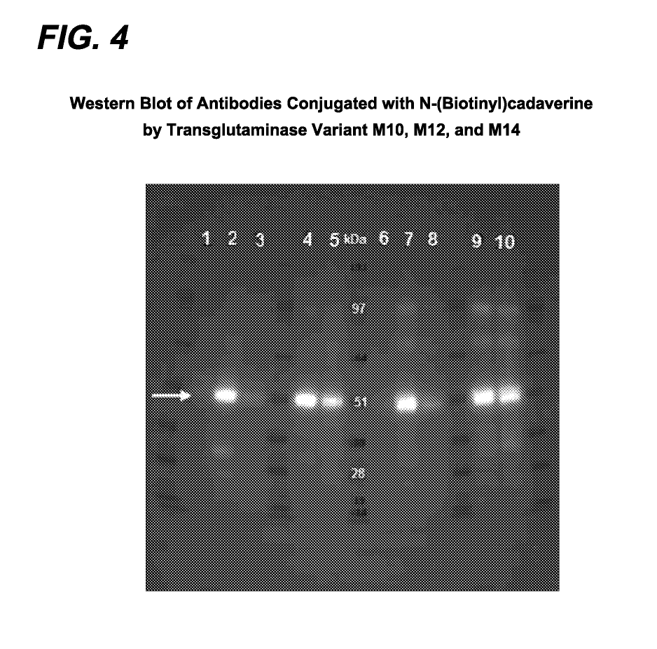

FIG. 4 is a western blot showing the results. The lane assignments are provided in Table 3.

TABLE 3
Conjugation of Antibodies to N-(Biotinyl)cadaverine by
Transglutaminase Varianwts M9, M10, M11, M12 and M14

| | | Transglutaminase |
|---|---|---|
| Lane | Antibody | Variant |
| 1 | Anti-glypican 3 | M9 |
| 2 | Anti-glypican 3 | M10 |
| 3 | Anti-glypican 3 | M11 |
| 4 | Anti-glypican 3 | M12 |
| 5 | Anti-glypican 3 | M14 |
| 6 | Anti-mesothelin | M9 |
| 7 | Anti-mesothelin | M10 |
| 8 | Anti-mesothelin | M11 |
| 9 | Anti-mesothelin | M12 |
| 10 | Anti-mesothelin | M14 |

Referring to the 51 kDa band, lanes 1, 3, 6, and 8, belonging to comparative variants M9 and M11 are dark, indicating that NBC was not present, while lanes 2, 4, 5, 7, 9, and 10, belonging to variants M10, M12, and M14 of this invention, were bright, indicating that biotin was attached and was detected and visualized by the NeutrAvidin Horseradish Peroxidase Conjugate.

The biotin/antibody ratios are shown in Table 4.

TABLE 4
Biotin to Antibody Ratio

| | Transglutaminase | Biotin/Antibody |
|---|---|---|
| Antibody | Variant | Ratio |
| Anti-glypican 3 | M10 | 0.55 |
| Anti-glypican 3 | M12 | 0.79 |
| Anti-glypican 3 | M14 | 0.69 |
| Anti-mesothelin | M10 | 0.70 |
| Anti-mesothelin | M12 | 0.64 |
| Anti-mesothelin | M14 | 0.91 |

### Example 5—Specific Activity of Variants M8, M10, M12, and M14

The specific activities of variants M8, M10, M12, and M14, compared to a BTG control (unmutated) are provided in Table 5. The activities were obtained using the Zedira kit referenced above and the substrate pair Z-Gln-Gly and hydroxylamine.

TABLE 5
Specific Activity of Transglutaminase Variants

| | Concentration | Specific Activity |
|---|---|---|
| Transglutaminase | (mg/mL) | (U/mg) |
| Control | 0.04 | 8.8 |
| Variant M8 | 0.11 | 5.3 |
| Variant M10 | 0.09 | 6.9 |
| Variant M12 | 0.09 | 4.5 |
| Variant M14 | 0.09 | 8.8 |

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

### REFERENCES

Full citations for the following references cited in abbreviated fashion by first author (or inventor) and date earlier in this specification are provided below. Each of these references is incorporated herein by reference for all purposes.
Bregeon et al., US 2013/0189287 A1 (2013).
Bregeon, WO 2014/202773 A1 (2014).
Bregeon et al., WO 2014/202775 A1 (2014).
Chen et al., US 2005/0136491 A1 (2005).
Dennler et al., Bioconjug. Chem. 2014, 25, 569.
Dorywalska et al., Bioconjug. Chem. 2015, 26, 650.
Fischer et al., WO 2014/072482 A1 (2014).
Fontana et al., Adv. Drug Deliv. Rev. 2008, 60, 13.
Hu et al., US 2009/0318349 A1 (2009).
Hu et al., US 2010/0087371 A1 (2010) [2010a].
Hu et al., US 2010/0099610 A1 (2010) [2010b].
Hu et al., WO 2015/191883 A1 (2015).
Innate Pharma, “A New Site Specific Antibody Conjugation Using Bacterial Transglutaminase,” presentation at ADC Summit, San Fransisco, Calif., Oct. 15, 2013.
Jeger, Doctoral Thesis, ETH Zurich, “Site-Specific Conjugation of Tumour-Targeting Antibodies Using Transglutaminase” (2009).
Jeger et al., Angew. Chem. Int. Ed. 2010, 49, 9995.
Kamiya et al., US 2011/0184147 A1 (2011).
Lhospice et al., Mol. Pharmaceutics 2015, 12, 1863.
Lin et al., J. Am. Chem. Soc. 2006, 128, 4542-4543.
Mero et al., Bioconjug. Chem. 2009, 384-389.
Mindt et al., Bioconjug. Chem. 2008, 19, 271.
Norskov-Lauritsen et al., US 2009/0117640 A1 (2009).
Pons et al., US 2013/0230543 A1 (2013).
Rao-Naik, U.S. Provisional Appl'n Ser. No. 62/130,673, filed Mar. 7, 2015.
Sato et al., U.S. Pat. No. 6,322,996 B1 (2001).
Sato, Adv. Drug Deliv. Rev. 2002, 54, 487.
Schibli et al., US 2007/0184537 A1 (2007).
Schrama et al., Nature Rev. Drug Disc. 2006, 5, 147.
Strop et al., Chemistry & Biology 2013, 20, 161.
Sugimura et al., J. Biotechnol. 2007, 131, 121.
Tagami et al., Protein Engineering Design Selection 2009, 22 (12), 747.
Yokoyama et al., Appl. Microbiol. Biotechnol. 2010, 87, 2087.

### Table of Sequences

TABLE 6
Sequence Summary

| SEQ ID NO: | SEQUENCE DESCRIPTION |
|---|---|
| 1 | S. mobaraensis BTG a.a. |
| 2 | Recombinant S. mobaraensis BTG proenzyme a.a. |
| 3 | Activated recombinant S. mobaraensis BTG a.a. |
| 4 | Variant M8 a.a. |
| 5 | Variant M10 a.a. |
| 6 | Variant M12 a.a. |
| 7 | Variant M14 a.a. |
| 8 | N-terminal tetrapeptide a.a. |
| 9 | Trypsin digest fragment a.a. |
| 10 | Primer zg67,901 n.t |
| 11 | Primer zg67,900 n.t. |
| 12 | Variant M8 amplicon n.t. |

What is claimed is:

1. A method of making an antibody conjugate, comprising:
(a) mixing an antibody with an amine donor compound comprising a primary amine and a moiety selected from the group consisting of a protein, a radioisotope, an assay agent, and a drug, in the presence of a variant transglutaminase comprising an amino acid sequence that is at least 90% identical to SEQ ID NO:1, with the proviso that the variant transglutaminase has an amino acid substitution feature selected from the group consisting of (A) E300A, (B) I240A and P241A, (C) E249Q, and (D) E300A and Y302A; and
(b) allowing the variant transglutaminase to catalyze the formation of an amide bond between the side chain carboxamide of a glutamine of the antibody and the primary amine of the amine donor compound, thereby making the antibody conjugate.

2. A method according to claim 1, wherein the antibody is an IgG antibody having a glutamine at position 295 and a glycosylated asparagine at position 297 (numbering per the EU index as in Kabat).

3. A method according to claim 1, wherein the variant transglutaminase has an amino acid substitution feature selected from the group consisting of (B) I240A and P241A, (C) E249Q, and (D) E300A and Y302A.

4. A method according to claim 1, wherein the moiety in the amine donor compound is a drug, preferably a DNA alkylator, tubulysin, auristatin, enediyne, pyrrolobenzodiazepine, or maytansinoid compound.

5. A method according to claim 1, wherein the amine donor compound has a structure represented by formula (I)
H₂N—(CH₂)₂₋₆D (I)
where D is a protein, a radioisotope, an assay agent, or a drug.

6. A method according to claim 1, wherein the amine donor compound has a structure represented by formula (Ia) 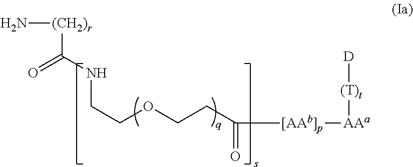
wherein
D is a drug, preferably a DNA alkylator, tubulysin, auristatin, enediyne, pyrrolobenzodiazepine, or maytansinoid compound;
T is a self-immolating group;
t is 0 or 1;
AA^{a} and each AA^{b} are independently selected from the group consisting of alanine, β-alanine, γ-aminobutyric acid, arginine, asparagine, aspartic acid, γ-carboxyglutamic acid, citrulline, cysteine, glutamic acid, glutamine, glycine, histidine, isoleucine, leucine, lysine, methionine, norleucine, norvaline, ornithine, phenylalanine, proline, serine, threonine, tryptophan, tyrosine, and valine;
p is 1, 2, 3, or 4;
q is 1, 2, 3, 4, 5, 6, 7, 8, 9, or 10;
r is 1, 2, 3, 4, or 5; and
s is 0 or 1.

7. A method of making an antibody conjugate, comprising:
(a) mixing an antibody with a first compound, which first compound is an amine donor compound having a primary amine and a first reactive functional group, in the presence of a variant transglutaminase comprising an amino acid sequence that is at least 90% identical to SEQ ID NO:1, with the proviso that the variant transglutaminase has an amino acid substitution feature selected from the group consisting of (A) E300A, (B) I240A and P241A, (C) E249Q, and (D) E300A and Y302A;
(b) allowing the variant transglutaminase to catalyze the formation of an amide bond between the side chain carboxamide of a glutamine of the antibody and the primary amine of the first compound, to make an adduct of the antibody and the first compound;
(c) contacting the adduct with a second compound having a second reactive functional group and a moiety selected from the group consisting of a protein, a radioisotope, an assay agent, and a drug; the second reactive functional group being capable of reacting with the first reactive functional group to form a covalent bond therebetween; and
(d) allowing the first and second reactive functional groups to react and form a covalent bond therebetween, thereby making the antibody conjugate.

8. A method according to claim 7, wherein the antibody is an IgG antibody having a glutamine at position 295 and a glycosylated asparagine at position 297 (numbering per the EU index as in Kabat).

9. A method according to claim 7, wherein the variant transglutaminase has an amino acid substitution feature selected from the group consisting of (B) I240A and P241A, (C) E249Q, and (D) E300A and Y302A.

10. A method according to claim 7, wherein the first compound has a structure represented by formula (II)
H₂N—(CH₂)₂₋₈—R′ (II)
wherein
R′ is selected from 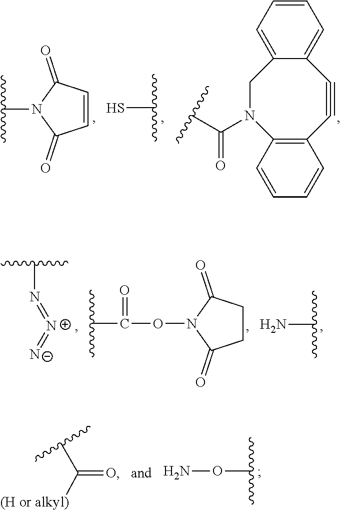
and the second compound has a structure represented by formula (III) 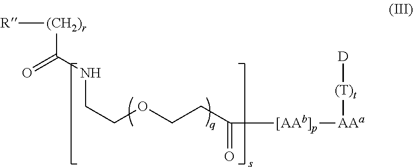
wherein
R″ is selected from 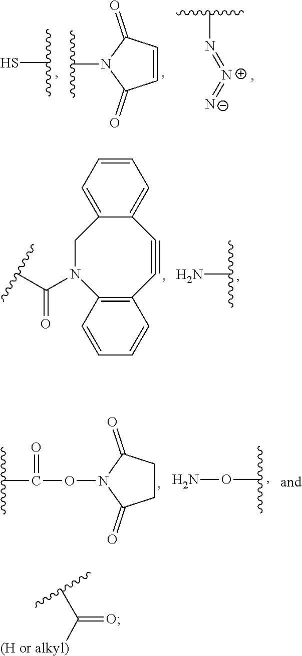
D is a drug that preferably is a DNA alkylator, tubulysin, auristatin, enediyne, pyrrolobenzodiazepine, or maytansinoid compound;
T is a self-immolating group;
t is 0 or 1;
AA^{a} and each AA^{b} are independently selected from the group consisting of alanine, β-alanine, γ-aminobutyric acid, arginine, asparagine, aspartic acid, γ-carboxyglutamic acid, citrulline, cysteine, glutamic acid, glutamine, glycine, histidine, isoleucine, leucine, lysine, methionine, norleucine, norvaline, ornithine, phenylalanine, proline, serine, threonine, tryptophan, tyrosine, and valine;
p is 1, 2, 3, or 4;
q is 1, 2, 3, 4, 5, 6, 7, 8, 9, or 10;
r is 1, 2, 3, 4, or 5; and
s is 0 or 1.

11. A variant transglutaminase comprising an amino acid sequence that is at least 95% identical to SEQ ID NO:1, with the proviso that the variant transglutaminase has an amino acid substitution feature selected from the group consisting of (a) I240A and P241A, (b) E249Q, and (c) E300A and Y302A.

12. A variant transglutaminase according to claim 11, having an I240A and a P241A amino acid substitution feature and comprising the amino acid sequence of SEQ ID NO:5.

13. A variant transglutaminase according to claim 11, having an E249Q amino acid substitution feature and comprising the amino acid sequence of SEQ ID NO:6.

14. A variant transglutaminase according to claim 11, having an E300A and a Y302A amino acid substitution feature and comprising the amino acid sequence of SEQ ID NO:7.
